(19) United States
(12) Patent Application Publication
(10) Pub. No.: US 2025/0221611 A1
(43) Pub. Date: Jul. 10, 2025

(54) NEEDLE SENSOR DERIVED IMAGE PLANE

(71) Applicant: INTUITIVE SURGICAL OPERATIONS, INC., Sunnyvale, CA (US)

(72) Inventors: Randall L. Schlesinger, San Mateo, CA (US); Serena H. Wong, Los Altos, CA (US); Samuel Raybin, San Jose, CA (US)

(21) Appl. No.: 18/852,126

(22) PCT Filed: Mar. 28, 2023
(86) PCT No.: PCT/US2023/065060

§ 371 Date: Sep. 27, 2024

Related U.S. Application Data
(60) Provisional application No. 63/325,082, filed on Mar. 29, 2022.

(51) Int. Cl. A61B 1/005; A61B 1/018; A61B 8/00; A61B 8/08

(52) U.S. Cl. CPC A61B 1/005; A61B 1/018; A61B 8/0841; A61B 8/445; A61B 8/4488; A61B 8/461

(57) ABSTRACT

A system includes a flexible elongate device, a tool, and a controller. The flexible elongate device includes a working channel extending to an opening in a distal portion of the flexible elongate device and a plurality of imaging elements. The tool is extendable through the working channel and the opening. The controller has one or more processors configured to determine a position associated with a portion of the tool, and control at least a portion of the plurality of imaging elements to generate an image that includes an imaging plane that includes the position associated with the portion of the tool.

### CROSS-REFERENCED APPLICATIONS

This application claims priority to and benefit of U.S. Provisional Application No. 63/325,082, filed Mar. 29, 2022 and entitled “Needle Sensor Derived Image Plane,” which is incorporated by reference herein in its entirety.

### FIELD

The present disclosure is directed to systems and methods for planning and performing an image-guided procedure.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert minimally invasive medical tools to reach a target tissue location. Minimally invasive medical tools include instruments such as therapeutic, diagnostic, biopsy, and surgical instruments. Medical tools may be inserted into anatomic passageways and navigated toward a region of interest within a patient anatomy. Navigation may be assisted using optical or ultrasound images of the anatomic passageways and surrounding anatomy, obtained pre-operatively and/or intra-operatively. Intra-operative imaging of a tool from a probe or catheter through which the tool is inserted may provide improved navigational guidance and confirmation of engagement of the tool with the target tissue.

Improved systems and methods are needed to efficiently track a tool location with respect to a catheter or probe through which the tool is inserted to improve imaging of the tool.

### SUMMARY

Consistent with some examples, a system may include a flexible elongate device, a tool, and a controller. The flexible elongate device may include a working channel extending to an opening in a distal portion of the flexible elongate device and a plurality of imaging elements. The tool may be extendable through the working channel and the opening. The controller may have one or more processors configured to determine a position associated with a portion of the tool, and control at least a portion of the plurality of imaging elements to generate an image that includes an imaging plane that includes the position associated with the portion of the tool. In some examples, the portion of the tool may include a distal tip of the tool.

In some examples, the plurality of imaging elements may include an annular array of the imaging elements disposed around the opening. The annular array may include at least two rows of imaging elements. In some examples, the plurality of imaging elements may include an array of ultrasound transmitters configured to generate ultrasound waves and an array of ultrasound sensors configured to detect reflected ultrasound waves. The ultrasound sensors may include whisper gallery mode (WGM) resonators. In some examples, the plurality of imaging elements may include an array of transducers configured to generate and detect ultrasound waves. In some examples, the plurality of imaging elements may include a grid array in which the plurality of imaging elements are arranged in at least two rows and at least two columns.

In some examples, the plurality of imaging elements may be configured to capture images including imaging planes defined within an imaging field located distally of the distal portion of the flexible elongate device. The working channel may extend through a distal end surface of the flexible elongate device to direct the tool into the imaging field. In some examples, the plurality of imaging elements may be configured to capture images including imaging planes defined within in an imaging field located radially outward from an outer side surface of the flexible elongate device. The working channel may extend through a side wall of the flexible elongate device to direct the tool into the imaging field.

In some examples, the controller may be further configured to determine a second position associated with one of a second position of the tool or a region of interest in a target tissue. The imaging plane may include both the position and the second position.

In some examples, the flexible elongate device may further comprise a balloon configured for inflation with a fluid for acoustic transmission between the plurality of imaging elements and tissue.

In some examples, the tool may comprise at least one of a biopsy needle, a therapeutic-delivery needle, an ablation device, or a cryogenic device. At least the portion of the tool may be flexible. The tool may include a localization sensor located at the portion of the tool. The localization sensor may comprise a fiber optic sensor configured to detect a change in a wavelength of light in a fiber in response to pressure waves generated by the plurality of imaging elements. The localization sensor may comprise a WGM resonator. The localization sensor may comprise a piezoelectric sensor. The localization sensor may comprise a capacitance micromachined ultrasound transducer.

In some examples, the controller may be configured to determine the position of the portion of the tool by sequentially activating a subset of the plurality of imaging elements to transmit pressure waves, detecting a pressure wave from each imaging element of the subset, determining a time of travel of the pressure wave from each imaging element of the subset to the localization sensor, and triangulating the position of the portion of the tool based on the time of travel of each pressure wave from the respective imaging element to the localization sensor.

In some examples, the plurality of imaging elements may be arranged in an annular array and the subset may comprise three imaging elements of the plurality of imaging elements. The three imaging elements may be spaced around a perimeter of the annular array with 120° of separation.

In some examples, the controller may be configured to determine the position of the portion of the tool by activating the localization sensor to transmit a pressure wave, activating a subset of the plurality of imaging elements to detect the pressure wave, determining a time of travel of the pressure wave from the localization sensor to each imaging element of the subset, and triangulating the position of the portion of the tool based on the time of travel of the pressure wave from the localization sensor to each imaging element of the subset.

In some examples, the controller may be configured to control the at least a portion of the plurality of imaging elements to generate the image by determining a firing sequence of the at least a portion of the plurality of imaging elements based on the imaging plane. The firing sequence may include a timing delay between successively firing imaging elements. The timing delay may be determined based on a geometry of an imaging field of the image with respect to a position of the plurality of imaging elements.

In some examples, the controller may be further configured to associate sets of imaging elements with different image planes, determine the imaging plane as being nearest to the position associated with the portion of the tool, and generate the image using a set of imaging elements associated with the determined imaging plane.

Consistent with some examples, a method for capturing an image performed by a controller including one or more processors may include determining a position associated with a portion of a tool. The tool may be at least partially located in a flexible elongate device. The flexible elongate device may include a plurality of imaging elements and a working channel extending to an opening in a distal portion of the flexible elongate device through which the portion of the tool extends. The method may further include controlling at least a portion of the plurality of imaging elements to generate an image that includes an imaging plane that includes the position associated with the portion of the tool.

In some examples, the method may include displaying a virtual indicator providing a visual representation of a position of the portion of the tool with respect to the imaging plane. The virtual indicator may include a distance indication providing a visual representation of a distance between the portion of the tool and the imaging plane. The virtual indicator may include a direction indication providing a representation of a direction of the portion of the tool with respect to the imaging plane. The direction indication may include a color in which at least a portion of the virtual indicator is displayed.

Other examples include corresponding computer systems, apparatus, and computer programs recorded on one or more computer storage devices, each configured to perform the actions of the methods.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 illustrates an example of an imaging probe in a patient anatomy near a target.

FIG. 2A illustrates an example of an imaging probe in accordance with the present disclosure.

FIG. 2B illustrates an example of an imaging array as shown in FIG. 2A.

FIG. 3A illustrates another example of an imaging probe in accordance with the present disclosure.

FIG. 3B illustrates an example of an imaging array as shown in FIG. 3A.

FIG. 3C illustrates an alternative example of an imaging array as shown in FIG. 3B.

FIGS. 3D-3H illustrate alternative examples of arrangements of components in an imaging probe.

FIG. 4A illustrates another example of an imaging probe in accordance with the present disclosure.

FIG. 4B illustrates an example of an imaging array as shown in FIG. 4A.

FIG. 5A illustrates an example of an imaging probe positioned within a catheter in accordance with the present disclosure.

FIG. 5B illustrates an example of an imaging array as shown in FIG. 5A.

FIG. 6A illustrates another example of an imaging probe positioned within a catheter in accordance with the present disclosure.

FIG. 6B illustrates an example of an imaging array as shown in FIG. 6A.

FIG. 6C illustrates another example of an imaging array as shown in FIG. 6A

FIG. 6D illustrates another example of an imaging array as shown in FIG. 6A.

FIG. 6E illustrates another example of an imaging array as shown in FIG. 6A.

FIG. 7A illustrates an arrangement of an imaging probe, a tool, and an imaging plane.

FIG. 7B illustrates an example of an image generated based on the arrangement of FIG. 7A.

FIG. 8A illustrates another arrangement of an imaging probe, a tool, and an imaging plane.

FIG. 8B illustrates an example of an image generated based on the arrangement of FIG. 8A.

FIG. 9A illustrates an example of an arrangement for determining a location of a portion of a tool for selection of an imaging plane.

FIG. 9B illustrates an example of an image generated based on a selected imaging plane after determining a location of the portion as described in relation to FIG. 9A.

FIG. 10A illustrates a flowchart of an example of a method for selecting an imaging plane and capturing an imaging.

FIG. 10B illustrates a flowchart of another example of a method for selecting an imaging plane and capturing an imaging.

FIG. 11 illustrates a simplified diagram of a robot-assisted medical system according to some examples of the present disclosure.

FIG. 12 illustrates a simplified diagram of a medical instrument system according to some examples of the present disclosure.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

The techniques disclosed in this document may be used to enhance the workflow processes of minimally invasive procedures using intra-operative direct visualization of a tool, such as intra-operative ultrasound imaging. In some examples, imaging data from an imaging probe may be utilized to verify real-time accurate placement of a treatment or diagnostic tool within an anatomical target during a medical procedure. Although described in the context of an ultrasound imaging probe, it is contemplated that the systems and methods described herein may be applied to other imaging modalities without departing from the scope of the present disclosure. For example, an imaging probe may be used to provide direct visual guidance of a tool as the tool is delivered via a flexible elongate device (e.g., a catheter or imaging probe) into a target. Such a tool may include, but is not limited to, an ablation device, biopsy tool (e.g., needle), therapeutic delivery tool, cryogenic device, irrigation, suction, or any other suitable medical treatment tool.

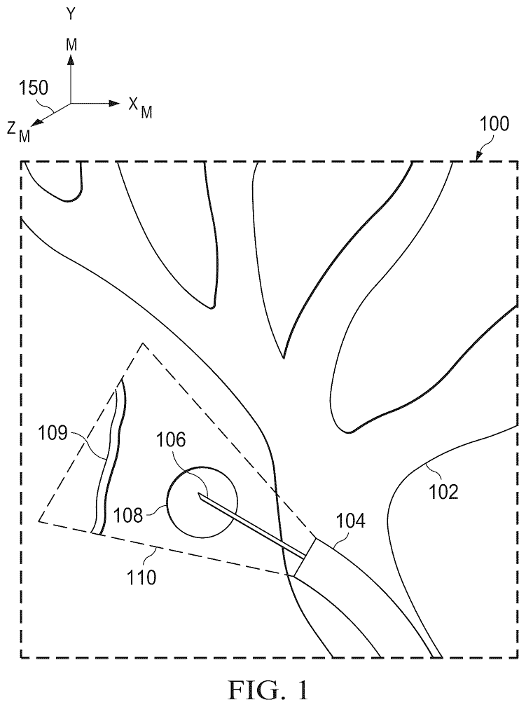

FIG. 1 illustrates an example of an image 100 including a flexible elongate device comprising an imaging probe 104 positioned within passageways 102 of a patient anatomy (which, as an example, may be airways of a lung) near a target tissue 108 which may be a lymph node, a suspected tumor, a nodule, or a lesion, in some examples. The image 100 may be displayed to a user on a graphical display and may be based on intra-operative imaging captured from an imaging system external to the patient (e.g., fluoroscopy, computed tomography, magnetic resonance imaging, etc.) or may be a virtual visualization based on a model of the patient anatomy generated from pre-operative or intra-operative image data. Image data may be captured and used to generate the three-dimensional model of anatomical structures of a patient, for example, by a control system. Imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like may be used to capture the model image data. For example, a CT scan of the patient's anatomy may be performed pre-operatively or intra-operatively and the resulting image data may be used to construct a 3D model which may be displayed to a user on a display system. The image 100 may present a global perspective of the anatomic model of the passageways 102 including a virtual representation of the imaging probe 104, including updating, based on data from a sensor system (e.g., sensor system 1508 of FIG. 15), a position and orientation of the imaging probe in real-time as it is navigated through the patient anatomy. In the illustrated example, the image 100 includes the passageways 102 based on a model of the patient anatomy in a model reference frame (X_{M}, Y_{M}, Z_{M}) 150 which may be registered to a surgical reference frame of the anatomy and/or an instrument reference frame of the imaging probe 104. For further description of registration between a model reference frame and an instrument reference frame or surgical reference frame, see U.S. Provisional Patent Application No. 63/240,471 filed Sep. 3, 2021 (disclosing “Ultrasound Elongate Instrument Systems And Methods”), which is incorporated herein by reference in its entirety for all purposes.

The imaging probe 104 may include one or more lumens for delivery of instruments, tools, devices, etc. In the illustrated example, a tool 106 comprising a needle has been delivered through a lumen of the imaging probe 104 into the target tissue 108. In some examples, the imaging probe 104 may include an imaging device for visualization of the tool 106, e.g., an ultrasound or optical imaging array, capturing images in an imaging field including an imaging plane 110. The imaging probe 104 includes a plurality (e.g., one or more arrays) of imaging elements. The imaging elements may be arranged to be any size, configuration, or shape. For example, the imaging elements may be arranged along a surface to form an array having a moon, ring, circle, or rectangle pattern. In some embodiments, the imaging elements include an array of transducers (e.g., lead zirconate titanate (PZT) transducers) that generate ultrasound waves and/or detect reflected ultrasound waves. In some embodiments, the imaging elements include an array of ultrasound receivers (e.g., whisper gallery mode (WGM) resonators) and an array of ultrasound transmitters (e.g., piezoelectric array). For additional description of an array of separate ultrasound receivers and ultrasound transmitters, see International Pat. Pub. No. WO 2021/119182 filed Dec. 9, 2020 (disclosing “Whispering Gallery Mode Resonators for Sensing Applications”), which is incorporated herein by reference in its entirety for all purposes.

The imaging probe 104 may generate images at various imaging planes based on control of the ultrasound transmission by the imaging elements. The transmitting imaging elements may use phased array ultrasonics or synthetic aperture techniques to capture images with selectable imaging planes. For a phased array ultrasonics, in order to direct the ultrasound energy in a desired direction to create a desired imaging plane, the imaging elements (e.g., transducers) are fired at a desired phase or time delay. In some examples, the phase or time delay can be calculated from a desired focal point/beam pattern. The firing sequence of the ultrasound imaging elements may be set to direct the ultrasound energy towards a target tissue. In some embodiments, different imaging planes that can be selected are associated with different configurations of the imaging elements. For example, various combinations and/or firing sequences of imaging elements may each be associated with one or more particular imaging planes. The control of the imaging elements to generate an image including the desired imaging plane may be based on the configuration and/or firing sequence associated with the desired imaging plane. A particular set of potential imaging planes may be preset and the associated combinations and/or firing sequences for each preset potential imaging plane may be stored in memory. In an example including an imaging array with imaging elements arranged radially around a central working channel, a preset set of potential imaging planes may be established at 5° intervals about an axis of the working channel. The physical arrangement of imaging elements may at least partially influence the spacing and orientation between the preset imaging planes. For example, an imaging array with a relatively high number of imaging elements in a dense arrangement may allow for a greater number of potential imaging planes than an imaging array with a relatively low number of imaging elements spaced apart. Upon determining a desired (or “optimum”) imaging plane as discussed further below, the controller may select the appropriate preset imaging plane of the set of preset imaging planes that most closely corresponds to the desired imaging plane. The controller may then activate the combination of imaging elements associated with the selected preset imaging plane in the firing sequence associated with the selected preset imaging plane. In some examples, one or more imaging element selection algorithms and/or firing sequence selection algorithms may be stored in memory and, upon determining the desired imaging plane, the controller may execute the one or more algorithms to select the appropriate imaging elements and/or firing sequence to generate an image along the desired imaging plane.

Since it can be beneficial to provide real time visualization of a tool positioned within a target, e.g., lesion, tumor, or nodule, to ensure accurate delivery of the tool within the target, the tool 106 may be delivered within the imaging plane 110 of the imaging probe 104 for direct visualization of the tool into the target tissue 108. Vasculature 109 is illustrated as being visualized within the imaging plane 110 in proximity to the target tissue 108, for example, by Doppler processing of ultrasound data. Identifying and avoiding vasculature 109 in the region of interest around the target tissue 108 may be particularly important in certain regions of the anatomy, such as in the mediastinum.

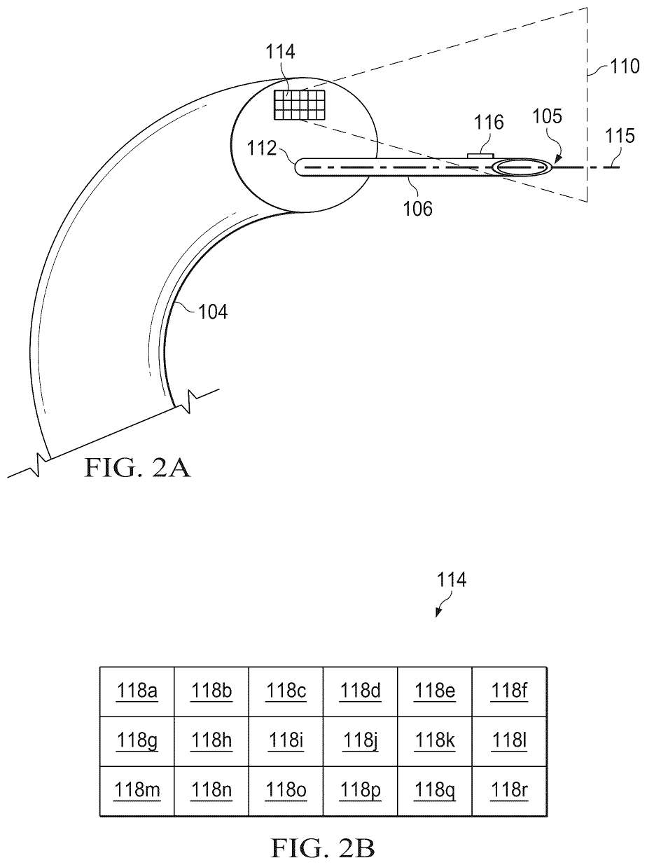

FIG. 2A illustrates an example of an imaging probe 104 including a forward-facing imaging array 114 configured to capture images in one or more imaging planes 110 taken through an imaging field disposed distally of the distal end of the imaging probe 104. In this regard, a tool 106 may be extended from an opening of a working channel 112 of the imaging probe 104 and into the imaging plane 110. The imaging array 114 may be configured to provide ultrasound images or any other suitable intraoperative imaging. It should be appreciated that the term “forward-facing” or “forward-viewing” as described herein encompasses imaging arrays with an imaging field that is directed at least partially forward (or distally) of the distal end of the device. This may include directly forward-facing imaging arrays which are oriented substantially parallel to the longitudinal axis of the instrument on which the imaging array is disposed, forward angled imaging arrays which have an imaging field angled with respect to the longitudinal axis, or the like. Accordingly, in some examples utilizing a forward-facing imaging array for delivery of a tool, the distal tip of the tool can be oriented with respect to the imaging array in a manner causing the tool to appear as a point, ellipse, or similar shape in the images. In order to visualize a larger portion of the tool, the distal tip of the imaging probe or portions of an imaging array can be angled, beveled, or shaped in a variety of ways to provide one or both direct axial and lateral images. In one example, the distal section of the imaging probe 104 supporting the imaging array 114 may be beveled or angled. In another example, the imaging array 114 may be transverse to the longitudinal axis of the imaging probe 104 but the working channel 112 may exit the distal tip at an angle, rather than parallel to the longitudinal axis. Accordingly, an angle may be formed between one or more imaging planes of the imaging array 114 and a longitudinal axis of the tool 106 and/or the working channel 112. Such an arrangement may reduce the likelihood of blocking the distal tip 105 of the tool with a more proximal portion of the tool. In an example, the angle between the imaging plane and tool may be approximately 2-30°, for example, 4°, 8°, or 12°. In the illustrated example of FIG. 2A, the imaging probe 104 has a forward-facing imaging array 114 that can collect images from a 3D volume distal of the array.

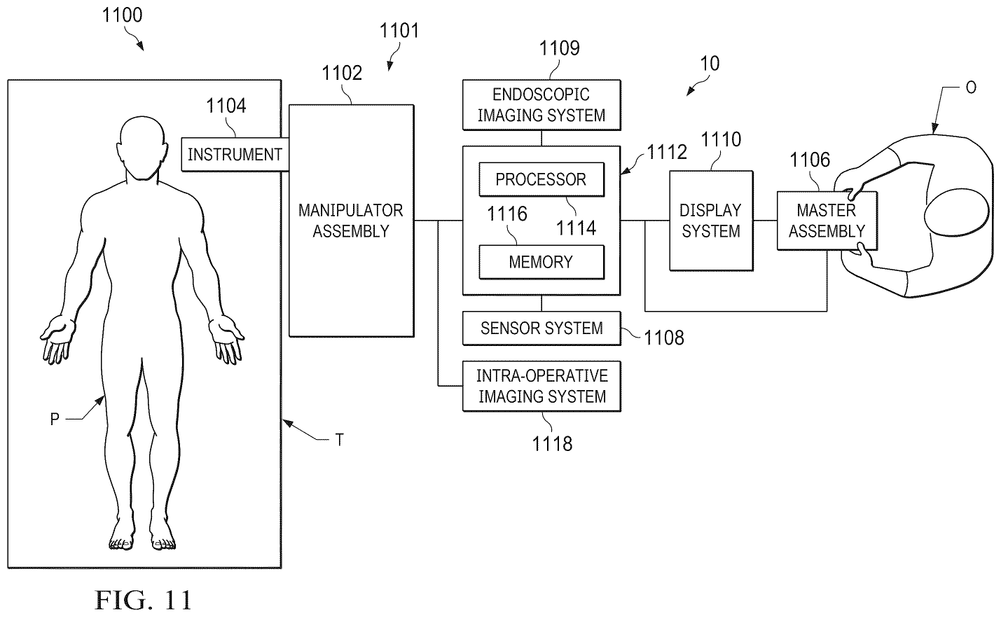

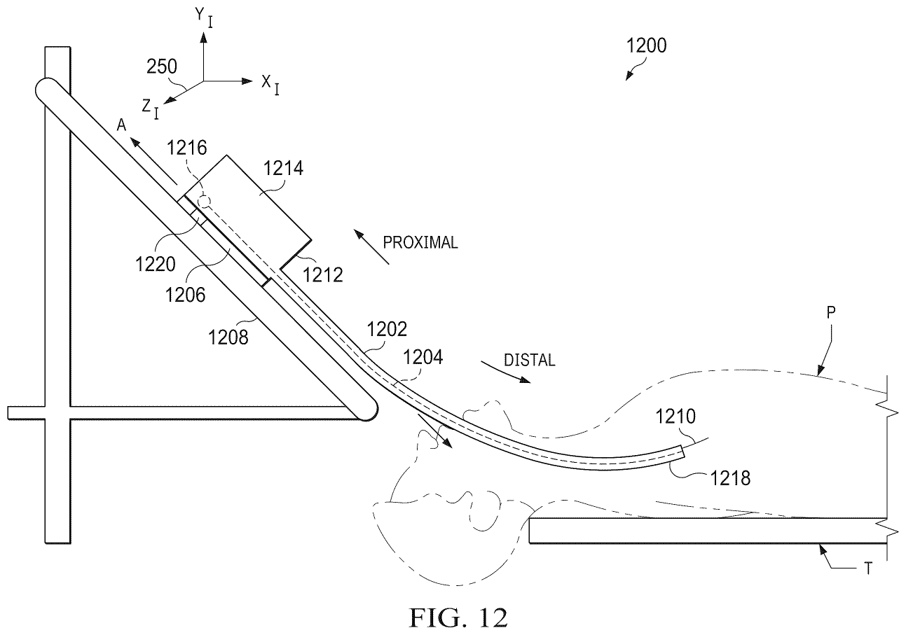

The imaging probe 104 may be steerable to navigate the imaging probe to a deployment location near the target tissue. For example, a plurality of pull wires or tendons may extend along a length of the imaging probe 104 and may be manipulated to steer the distal end of the imaging probe. Alternatively, the imaging probe 104 may be passively flexible and may be navigated to a deployment location by a steerable catheter or sheath having a lumen through which the imaging probe is disposed. In some examples, imaging probe 104 may include a lumen for receipt of a guidewire to navigate the imaging probe to a deployment location. Further discussion of navigation of an imaging probe 104 to a deployment location is provided below in relation to FIGS. 11 and 12.

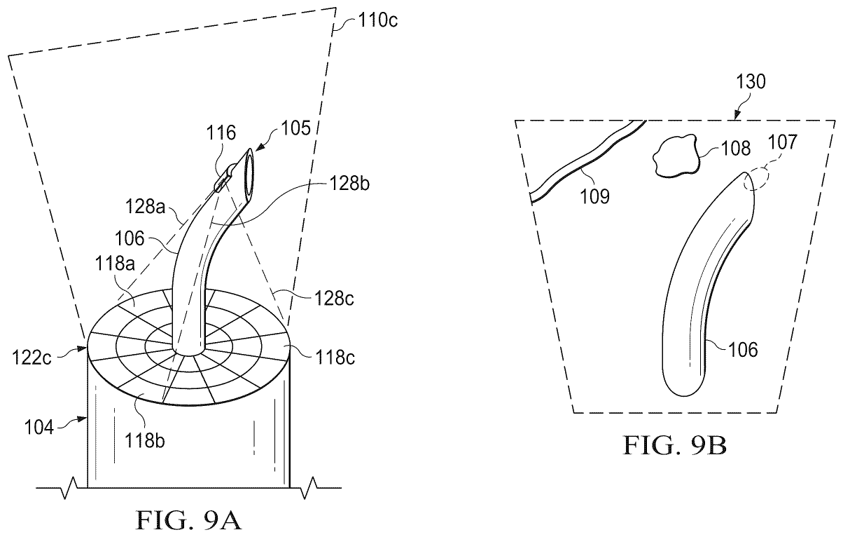

FIG. 2B provides a more detailed view of the imaging array 114 of FIG. 2A. In this example, the imaging array includes 18 imaging elements (e.g., transducers) arranged in a grid of three rows and six columns. A controller, having one or more processors and a memory, in communication with the imaging array 114 can control an activation order of the various imaging elements 118 to generate images in a plurality of imaging planes. In some examples, the controller may be configured generate a three-dimensional image data volume by aggregating image data from a plurality of imaging planes, similar to CT volumes constructed from a plurality of image slices. This process may be computationally burdensome and may result in undesirable time delays during a procedure. Accordingly, it may be desirable to omit such three-dimensional construction of a volume and instead utilize only a single imaging plane that is determined to be best suited for displaying the features of interest. Because the distal tip 105 of tool 106 is likely to be the feature of the tool that the user is most interested in visualizing, the controller may identify and select an optimum imaging plane 110 that will include the distal tip 105 or a portion of the tool 106 near the distal tip. In this regard, a localization sensor 116 may be disposed at or near the distal tip 105, in a fixed known relation thereto, for tracking a position of the distal tip. As described in more detail in relation to FIG. 9A below, a portion or subset of the imaging elements 118 may be selected and activated (or “controlled”) in an order determined by the controller. Activation of the imaging elements 118 of the subset, in conjunction with use of the localization sensor 116 to receive a signal (e.g., a pressure or acoustic wave) from the imaging elements of the subset, may allow the controller to determine a position of the localization sensor 116 in relation to the imaging array 114. Because the position of the localization sensor 116 is fixed with respect to the tool 106, a position of the distal tip 105 or other portion of the tool may also be determined. Although examples herein describe determining a location of a distal tip of the tool and generating an image having an imaging plane that includes the distal tip, it will be appreciated that any other portion of the tool that is exposed and within the imaging field of the imaging probe may be used in place of the distal tip.

Surface enhancements may be included on the tool 106 to facilitate imaging. For example, an external surface of the tool 106 may be coated, roughened, or include grooves or slits to affect a scatter pattern of a signal (e.g., pressure or acoustic wave) from the imaging elements. In some examples, such a surface enhancement may be provided on only a limited portion or portions of the tool 106. In an example, a portion of the tool 106 near or at the distal tip 105 may be smooth while a portion of the tool proximal of the distal tip may include a roughened surface to disperse imaging signals. In this regard, the portion including the distal tip 105 may appear more distinct in an image than a portion proximal thereof, providing visual confirmation that the distal tip 105 has been captured in the image(s). In an example, a portion of the tool 106 near or at the distal tip 105 may be coated in an echogenic material while a portion of the tool proximal of the distal tip may not. In this regard, the portion including the distal tip 105 may appear more distinct in an image than a portion proximal thereof, providing visual confirmation that the distal tip 105 has been captured in the image(s).

As will be appreciated, in the event that the tool 106 is rigid, it may be expected to extend distally from the opening of the working channel 112 directly along an axis 115 extending from the working channel. As such, the alignment of the tool 106 with respect to the imaging array 114 may be assumed based on a known location of the axis 115 of the working channel 112 with respect to the imaging array 114. In this regard, an optimum imaging plane, e.g., the imaging plane aligned with the axis 115, may be predetermined. However, in the event that the tool 106 is flexible, the tool may bend away from the axis 115 as it is extended from the imaging probe 104. For example, a flexible biopsy needle may bend away from the axis 115 as it encounters resistance from tissue between the imaging probe 104 and the target tissue. Accordingly, it will be appreciated that an imaging plane aligned with the working channel 112 may not always be suitable for displaying the distal tip of a flexible tool. Moreover, when a flexible tool bends out of plane, it may not be readily apparent when viewing an image from the imaging probe that the portion of the tool being displayed does not include the distal tip. Therefore, it may be desirable to use an alternative imaging plane laterally displaced from the axis 115 of the working channel 112 that may provide an optimum imaging plane based on the determined position of the distal tip 105.

After determining the position of the distal tip 105, the controller may identify another subset and a corresponding activation order of the imaging elements 118 suitable to generate an imaging plane 110 that includes the distal tip 105. For example, different imaging planes 110 are associated with different positions, and thus the determined position of the distal tip 105 may be used to select the appropriate imaging plane 110. In some examples, the spatial resolution of the potential imaging planes of the imaging array 114 may result in an inability of the imaging array to generate an imaging plane that captures the distal tip 105. That is, the potential imaging planes of the imaging array 114 may be spaced apart by some known distance (e.g., 0.25 mm) and the distal tip 105 may be determined to be positioned between two adjacent potential imaging planes. Accordingly, the controller may determine which potential imaging plane is nearest to the distal tip 105 and select that imaging plane as the optimum imaging plane 110. The controller may then activate the selected imaging elements 118 to generate an image in the optimum imaging plane 110. That is, the controller may control at least a portion of the plurality of imaging elements 118 to generate an image that includes an imaging plane that includes the position of the distal tip.

Advantageously, the imaging technique described herein may reduce the computational burden of imaging processing across a plurality of imaging planes by identifying the optimum imaging plane and capturing an image only in that plane. This may reduce the time required to construct and display an image to user, enhancing real-time visualization of a tool intra-operatively. The imaging plane may be selected to display any portion of the tool 106 of interest (e.g., not necessarily the distal tip 105), or some other feature (e.g., target tissue 108) of interest.

In some embodiments, an imaging plane 110 may be selected based on multiple positions or features of interest. For example, an imaging plane 110 that includes both the distal tip 105 of the tool 106 and a portion of the target tissue 108 may be selected. In another example, an imaging plane 110 that includes the distal tip 105 of the tool 106 and another (e.g., more proximal) portion of the tool 106 may be selected. As such, an optimal imaging plane 110 may be selected that includes multiple positions or features of interest.

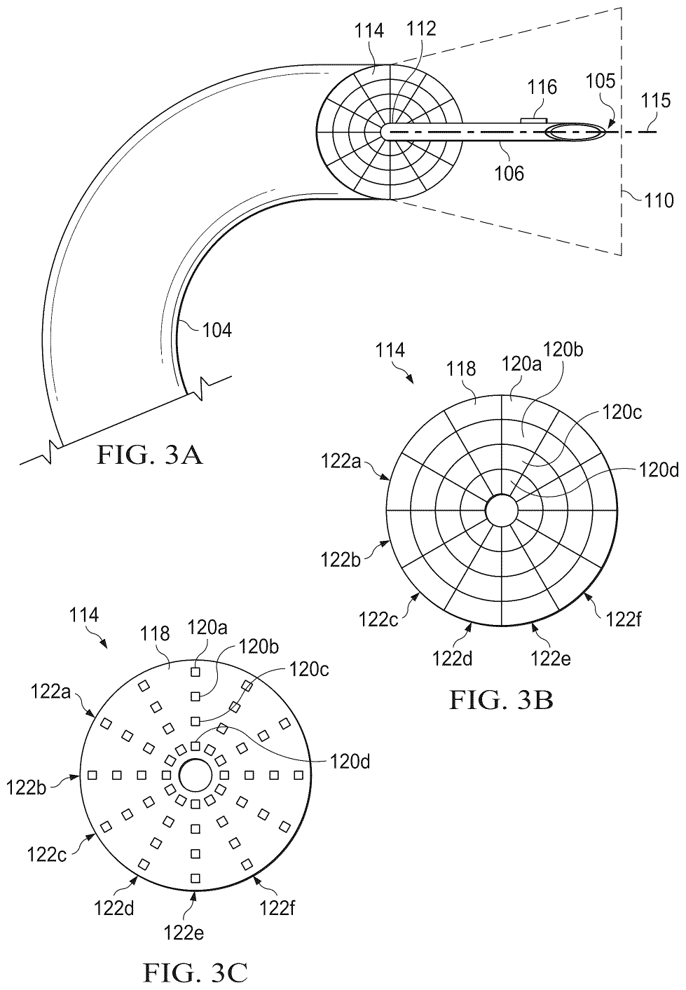

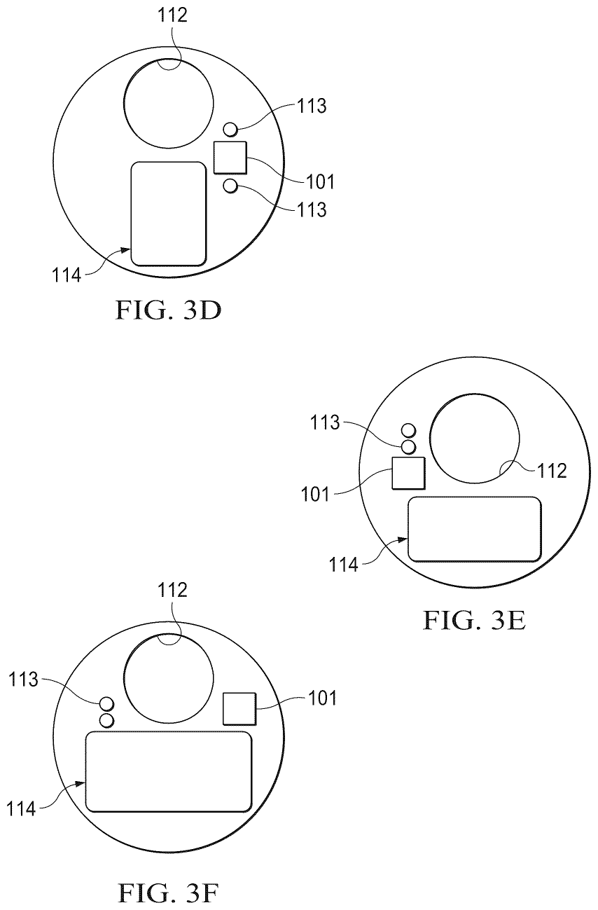

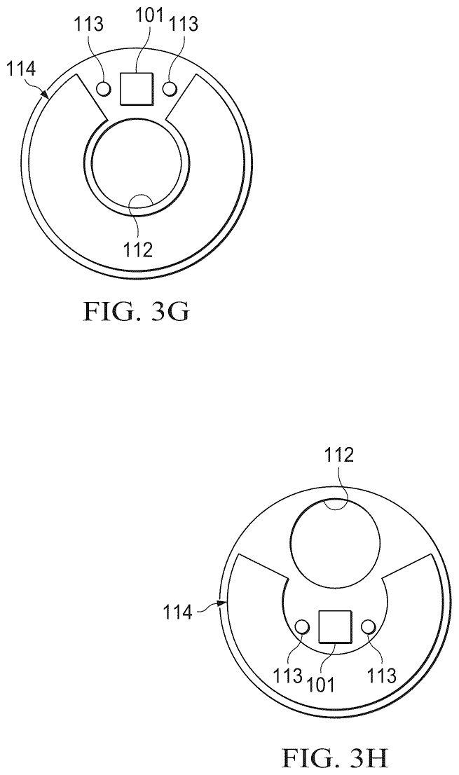

FIGS. 3A, 4A, 5A, and 6A illustrate examples of imaging probes 104 similar to that of FIG. 2A but with alternative structural arrangements. FIG. 3A illustrates an example of an imaging probe 104 including a forward-facing ring or annular shaped imaging array 114 disposed about a working channel 112 and configured to capture images in one or more imaging planes 110 taken through an imaging field disposed distally of the distal end of the imaging probe 104. In this regard, a tool 106 may be extended from a working channel 112 of the imaging probe 104 and into the imaging plane 110. The imaging probe 104 of FIG. 3A may be independently steerable similar to the imaging probe of FIG. 2A. FIG. 3B illustrates a simplified diagram of the imaging array 114 of FIG. 3A, including a plurality of imaging elements 118. The imaging elements 118 are arranged in a plurality of rows or rings 120a-120d and a plurality of columns 122a-122f, each column 122a-122f comprising eight imaging elements extending linearly across the width of the imaging array 114 in the illustrated example. It will be appreciated that although illustrated in FIGS. 3A and 3B as having an arcuate shape, each imaging element 118 may be square or rectangular as shown in FIG. 3C. Any suitable subsets of imaging elements 118 may be utilized to determine a position of the localization sensor 116 and distal tip 105 or to generate an image in an imaging plane 110 determined to be optimal based on the determined position of the localization sensor 116 and/or distal tip 105.

FIG. 3D illustrates a front view of an example of a distal end of an imaging probe 104. A working channel 112 is positioned in an offset position from a central longitudinal axis of the imaging probe. An imaging array 114 is positioned diametrically opposed to the working channel 112. In this example, the imaging array 114 is rectangular, although any suitable shape may be used, and may include any number of imaging elements (e.g., transducers) arranged in a grid of rows and columns, for example. An image sensor 117, such as a CCD or CMOS camera, is positioned laterally offset from the working channel 112 and imaging array 114. Light guides 113 are positioned on opposing sides of the image sensor 117. The light guides 113 may comprise an LED, a channel housing a fiber optic, or any other suitable light emitting device.

FIG. 3E illustrates a front view of an example of a distal end of an imaging probe 104. A working channel 112 is positioned in an offset position from a central longitudinal axis of the imaging probe. An imaging array 114 is positioned diametrically opposed to the working channel 112. In this example, the imaging array 114 is rectangular, although any suitable shape may be used, and may include any number of imaging elements (e.g., transducers) arranged in a grid of rows and columns, for example. An image sensor 117, such as a CCD or CMOS camera, is positioned laterally offset from the working channel 112 and imaging array 114. A pair of light guides 113 are positioned on one side of the image sensor 117. The light guides 113 may comprise an LED, a channel housing a fiber optic, or any other suitable light emitting device.

FIG. 3F illustrates a front view of an example of a distal end of an imaging probe 104. A working channel 112 is positioned in an offset position from a central longitudinal axis of the imaging probe. An imaging array 114 is positioned diametrically opposed to the working channel 112. In this example, the imaging array 114 is rectangular, although any suitable shape may be used, and may include any number of imaging elements (e.g., transducers) arranged in a grid of rows and columns, for example. An image sensor 117, such as a CCD or CMOS camera, is positioned laterally offset from the working channel 112 and imaging array 114 on one side and light guides 113 are positioned laterally offset from the working channel 112 and imaging array 114 on the other side. The light guides 113 may comprise an LED, a channel housing a fiber optic, or any other suitable light emitting device.

FIG. 3G illustrates a front view of an example of a distal end of an imaging probe 104. A working channel 112 is positioned along a central longitudinal axis of the imaging probe. An imaging array 114 extends in an arc around at least a portion of the working channel 112. In this example, the imaging array 114 extends through approximately 300°, although any suitable arc length may be used. For example, the imaging array 114 may extend through an arc of 60-350°, such as 90°, 180°, 270°, or 340°. The imaging array may include any number of imaging elements (e.g., transducers) arranged in a grid of rows and columns, for example. An image sensor 117, such as a CCD or CMOS camera, is positioned between opposing ends of the imaging array 114. A pair of light guides 113 are positioned on opposing sides of the image sensor 117. The light guides 113 may comprise an LED, a channel housing a fiber optic, or any other suitable light emitting device.

FIG. 3H illustrates a front view of an example of a distal end of an imaging probe 104. A working channel 112 is positioned offset from a central longitudinal axis of the imaging probe. An imaging array 114 is centered in a position diametrically opposed from the working channel 112 and extends in an arc. In this example, the imaging array 114 extends through approximately 340°, although any suitable arc length may be used. For example, the imaging array 114 may extend through an arc of 60-350°, such as 90°, 180°, 270°, or 340°. The imaging array may include any number of imaging elements (e.g., transducers) arranged in a grid of rows and columns, for example. An image sensor 117, such as a CCD or CMOS camera, is positioned within an interior of the arc of the imaging array 114. A pair of light guides 113 are positioned on opposing sides of the image sensor 117. The light guides 113 may comprise an LED, a channel housing a fiber optic, or any other suitable light emitting device.

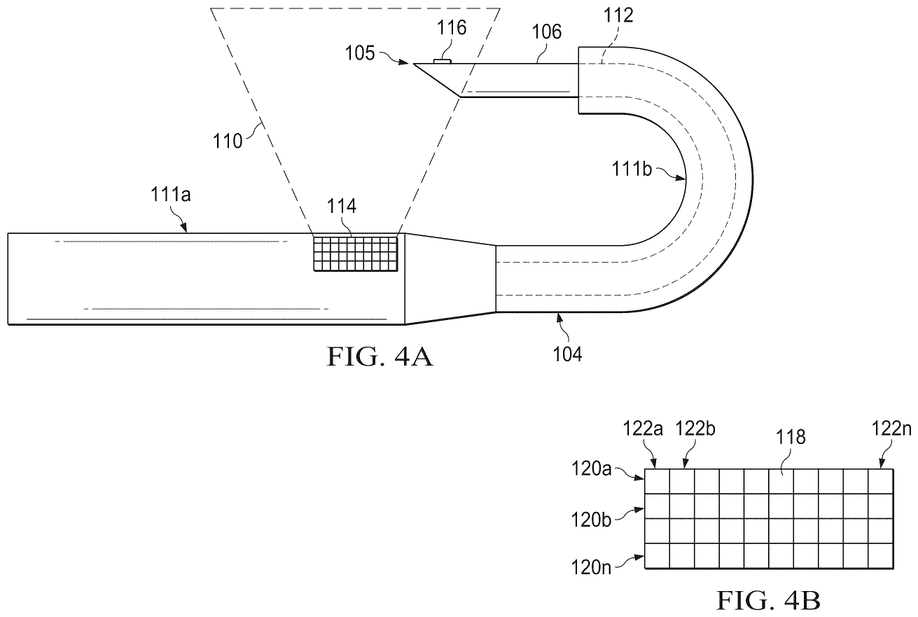

FIG. 4A illustrates an example of an imaging probe 104 including a side-facing imaging array 114 disposed on a side of the imaging probe and configured to capture images in one or more imaging planes 110 taken through an imaging field disposed radially outward from a side of the imaging probe 104. The imaging probe 104 of FIG. 4A may include a proximal portion 111a and a distal portion 111b which may be more flexible than the proximal portion. Examples of a flexible elongate instrument which may be similar to the imaging probe of FIG. 4A are described in U.S. patent application Ser. No. 16/632,128 filed Jan. 17, 2020 (disclosing “Flexible Elongate Device Systems and Methods”), which is incorporated herein by reference in its entirety for all purposes. A tool 106 may be extended from a working channel 112 of the distal portion 111b of the imaging probe 104 into the imaging plane 110. FIG. 4B illustrates a simplified diagram of the imaging array 114 of FIG. 4A, including a plurality of imaging elements 118. The imaging elements 118 are arranged in a plurality of rows 120a-120n and a plurality of columns 122a-122n. Any suitable subsets of imaging elements 118 may be utilized to determine a position of the localization sensor 116 and distal tip 105 or to generate an image in an imaging plane 110 determined to be optimal based on the determined position of the localization sensor 116 and/or distal tip 105.

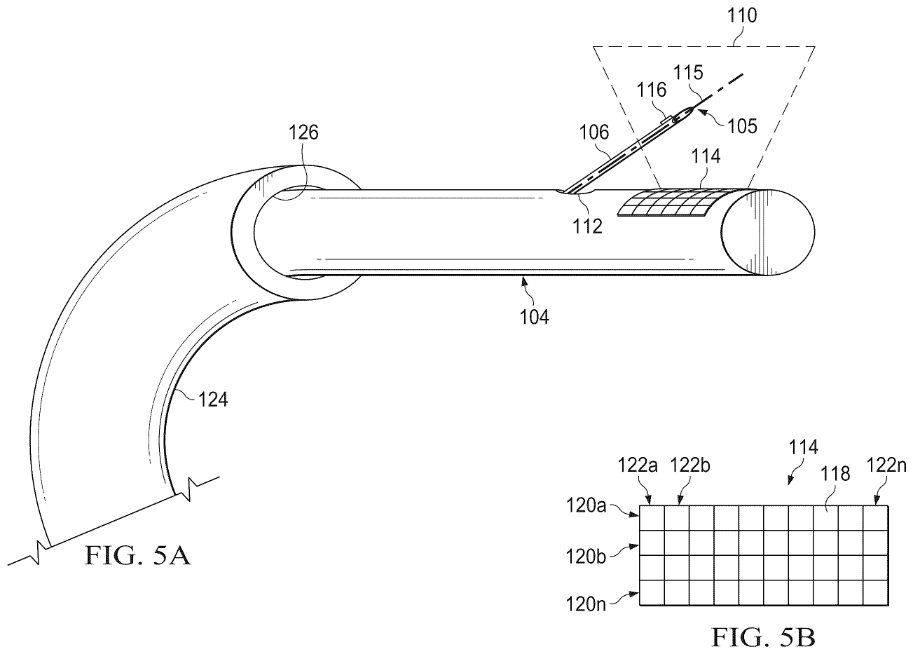

FIG. 5A illustrates an example of an imaging probe 104 including a side-facing or side-viewing imaging array 114 disposed distally of a working channel 112 extending through a side port and configured to capture images in one or more imaging planes 110 taken through an imaging field disposed radially outward from a side of the imaging probe 104. In this regard, a tool 106 may be extended from the working channel 112 and into the imaging plane 110. The imaging probe 104 is shown in FIG. 5A extending distally from a lumen 126 of a steerable catheter 124. The catheter 124 may be used to guide the imaging probe 104 to a deployment location within the patient anatomy. FIG. 5B illustrates a simplified diagram of the imaging array 114 of FIG. 5A, including a plurality of imaging elements 118. The imaging elements 118 are arranged in a plurality of rows 120a-120n and a plurality of columns 122a-122n. Any suitable subsets of imaging elements 118 may be utilized to determine a position of the localization sensor 116 and distal 105 or to generate an image in an imaging plane 110 determined to be optimal based on the determined position of the localization sensor 116 and/or distal tip.

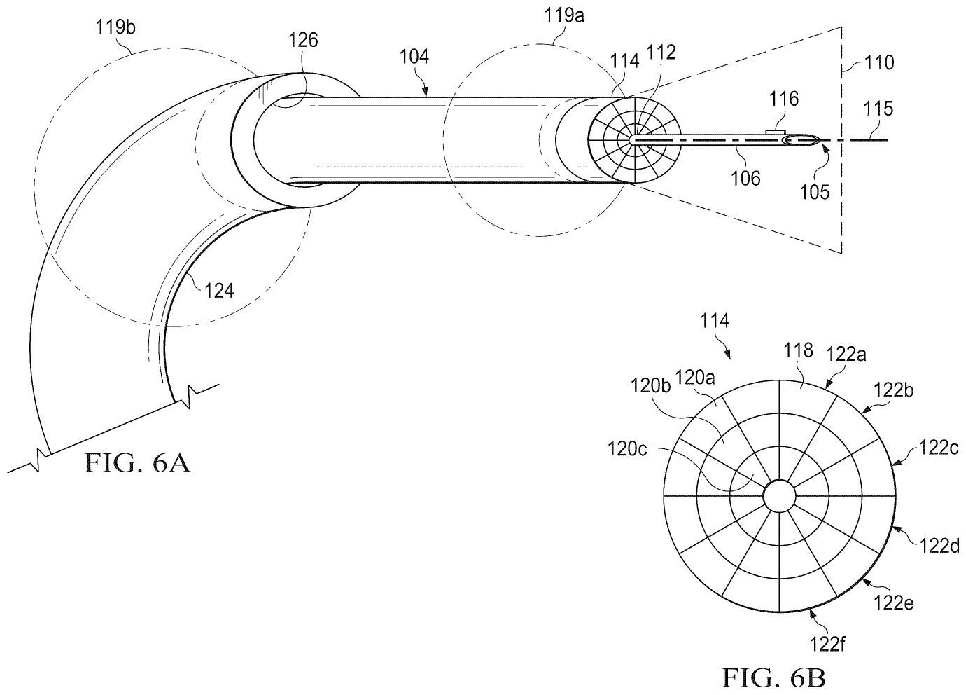

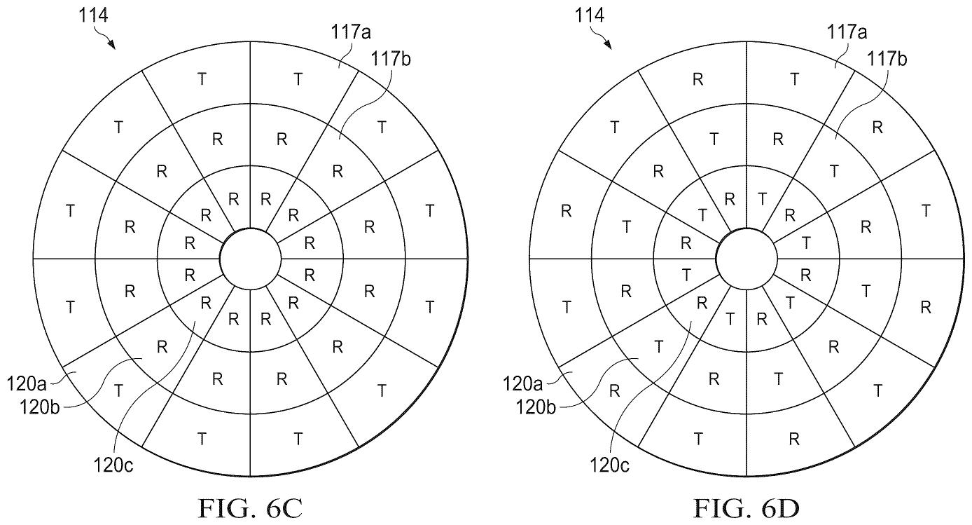

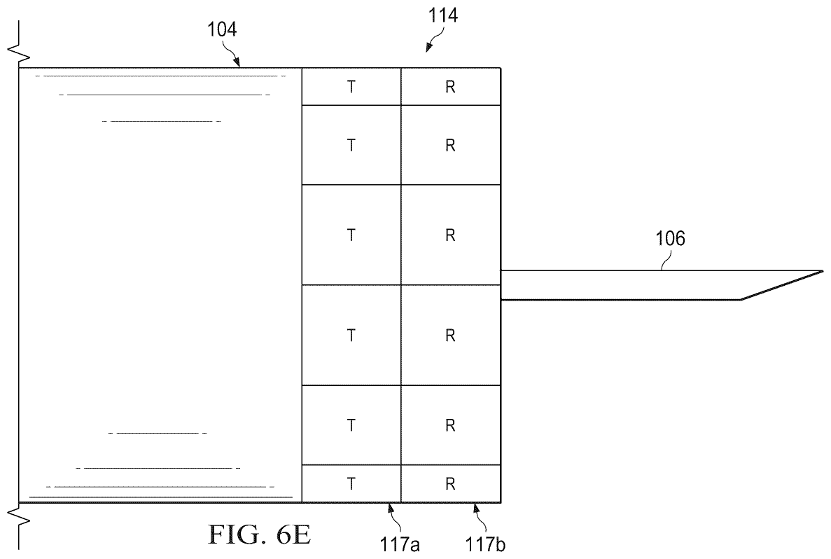

FIG. 6A illustrates an example of an imaging probe 104 including a forward-facing ring or annular shaped imaging array 114 disposed about a working channel 112 and configured to capture images in one or more imaging planes 110 taken through an imaging field disposed distally of the distal end of the imaging probe 104. In this regard, a tool 106 may be extended from a working channel 112 of the imaging probe 104 and into the imaging plane 110. The imaging probe 104 of FIG. 6A may be passively steerable using a catheter 124 having a lumen 126 through which the imaging probe 104 is disposed. FIG. 6B illustrates a simplified diagram of the imaging array 114 of FIG. 6A, including a plurality of imaging elements 118. The imaging elements 118 are arranged in a plurality of rows 120a-120c and a plurality of columns 122a-122f, each column including six imaging elements extending linearly across a width of the imaging array 114 in the illustrated example. Although shown as having an arcuate shape, it will be appreciated that each imaging element 118 of FIGS. 6A and 6B may have a square or rectangular shape, similar to the arrangement shown in FIG. 3C. Any suitable subsets of imaging elements 118 may be utilized to determine a position of the localization sensor 116 and distal tip 105 or to generate an image in an imaging plane 110 determined to be optimal based on the determined position of the localization sensor 116 and/or distal tip 105.

In some of the above-described examples, the imaging elements 118 may be transducers capable of both transmitting and detecting acoustic waves. In some examples, the imaging elements 118 may each be a piezoelectric transmitter or a resonator. In some examples, the imaging array includes a piezoelectric array configured to generate ultrasound waves. The imaging elements of the imaging array includes WGM resonators configured to detect reflected ultrasound waves. The WGM resonators may include optical fiber resonators, microsphere resonators, microbubble resonators, microbottle resonators, microtoroid resonators, microdisk resonators, microring resonators, or some other structure of resonators. WGM resonators measure the intensity of reflected ultrasound waves. For example, incoming ultrasound echoes change the resonant frequency of the WGM resonators to generate resonance shift, by modulating the refractive index of the material of the WGM resonators, or physically deforming the WGM resonators.

FIG. 6C illustrates a simplified diagram of an example of the imaging array 114 of FIG. 6A, including a plurality of piezoelectric transmitters 117a (designated “T”) and a plurality of WGM resonators 117b (designated “R”) or other type of acoustic sensor. Each transmitter 117a may be configured to generate and transmit an acoustic wave and each resonator 177b may be configured to detected reflected acoustic waves. In this illustrated example, the outer row 120a of imaging elements comprises transmitters 117a and the inner rows 120b, 120c comprise resonators 117b. It should be appreciated that any of the illustrated rows 120a-120b may comprise transmitters 117a or resonators 117b. For example, a single row of resonators may be disposed between two rows of transmitters.

FIG. 6D illustrates a simplified diagram of another example of the imaging array 114 of FIG. 6A, including a plurality of piezoelectric transmitters 117a (designated “T”) and a plurality of WGM resonators 117b (designated “R”). In this illustrated example, the transmitters 117a and resonators 117b are interspersed such that each row 120a-120c of imaging elements is formed by an alternating sequence of transmitters and resonators. Although illustrated as a 1:1 alternating pattern, any suitable pattern or arrangement of transmitters and resonators may be used.

FIG. 6E illustrates a simplified diagram of another example of the imaging array 114 of FIG. 6A, including a plurality of piezoelectric transmitters 117a (designated “T”) and a plurality of WGM resonators 117b (designated “R”). In this illustrated example, the transmitters 117a form a layer of imaging elements positioned proximally of (behind) a layer of resonators 117b. Alternately, a layer of transmitters could be positioned distally of a layer of resonators.

Although the examples of FIGS. 6C-6E are provided with reference to FIG. 6A, it should be appreciated that the concepts discussed in relation to these figures may similarly be applied to the imaging arrays 114 of any of FIGS. 2A-5B. For example, each of the imaging elements 118 of any of these figures may be illustrative of a transducer or of either a transmitter 117a or a resonator 117b. In this regard, the term “imaging array” as used herein may refer to a single array formed of a plurality of transducers or may refer to a plurality of arrays formed of a plurality of transmitters and a plurality of resonators.

As with the imaging probe 104 of FIG. 2A, a flexible tool 106 inserted through the imaging probes 104 of FIGS. 3A-6B may bend away from the axis 115 and out of plane with respect to one or more potential imaging planes of the imaging arrays 114. Accordingly, a position of the distal tip 105 of the tool 106 may be determined using a localization sensor 116 and a subset of the imaging elements 118 may be selected to provide an optimum imaging plane 110 to capture the distal tip 105 or other features of interest as described in relation to FIGS. 2A-2B.

It should be appreciated that the shapes, arrangements, sizes, and placement of the imaging arrays 114 of the illustrated examples may be adjusted as is suitable for any given implementation. That is, the figures should not be considered to be limiting with regard to the illustrated specific array shapes, specific number of imaging elements in the imaging arrays, the specific shapes of each imaging element, the spacing between adjacent imaging elements, etc. These illustrations are provided only as examples of the various configurations contemplated within the scope of the present disclosure.

It should further be appreciated that with ultrasound imaging, it is often desirable to secure the imaging probe in place for stabilization and minimize the volume of air and other gases between an imaging array and tissue which is to be imaged. Accordingly, a variety of balloons or inflatable/expandable fluid containing devices can be used to ensure fluid contact. For example, a balloon 119a may be disposed adjacent to or around the distal end of the imaging probe 104 and/or a balloon 119b may be disposed adjacent to the distal end of the catheter 124. A balloon 119 may be inflated with a coupling fluid to expand the balloon into contact with the surrounding tissue of an anatomical passageway to park or secure the imaging probe 104 in place and/or to fill the space between the imaging array and tissue that is to be imaged with a fluid that is conducive to imaging.

In some embodiments, an imaging array may be disposed within an inflatable balloon 119a and the inflatable balloon may be inflated with a fluid that is conducive to the applicable imaging medium, thereby reducing the volume of air or other gases between the imaging array and the patient's tissue. Inflating the balloon 119a in this manner may also park the imaging probe 104 to secure it in a desired location within the passageway. A balloon 119b may be disposed on catheter 124 to aid in parking the catheter.

In some examples, an inflatable balloon 119 may also be used to seal an anatomical passageway for suction or insufflation through a device such as the imaging probe 104 or catheter 124. In this regard, a balloon 119 may be used to block a passageway so that air may be suctioned from a portion of the passageway distal to the device, thereby collapsing the passageway. As an alternative to collapsing the passageway, a fluid or imaging gel may be injected into a portion of the passageway distal to the device after inflating the balloon, thereby filling the portion of the passageway with a medium that is conducive to imaging. Collapsing the passageway or filling the passageway with fluid as described may eliminate or reduce any volume of air which otherwise may hinder imaging quality.

In some examples, a balloon 119 may be used to retain an imaging array in direct contact with tissue. For example, in the case of a side-facing imaging array, a balloon on one side of an imaging probe may be inflated to push the imaging probe laterally into contact with tissue. In this regard, an imaging array on an opposing side of the imaging probe from the balloon may be forced into direct contact with the tissue. In another example, in the case of a forward-facing imaging array, the imaging device at the distal end of the imaging probe may be driven into direct contact with tissue to be imaged. Then a balloon 119a extending radially around the imaging probe may be inflated into contact with surrounding tissue to secure the imaging probe in place.

In this regard, it should be appreciated that an inflatable balloon may be beneficial for use with any forward-facing imaging array or side-facing imaging array discussed herein.

Additional description of examples of a flexible elongate instrument which may be similar to the imaging probes discussed herein and/or an inflatable balloon for facilitating imaging are described in U.S. Provisional Patent Application No. 63/240,471 filed Sep. 3, 2021 (disclosing “Ultrasound Elongate Instrument Systems And Methods”), which is incorporated herein by reference in its entirety for all purposes. Although illustrated only in FIG. 6A, the balloons 119 may be similarly applicable to any example of an imaging probe described herein.

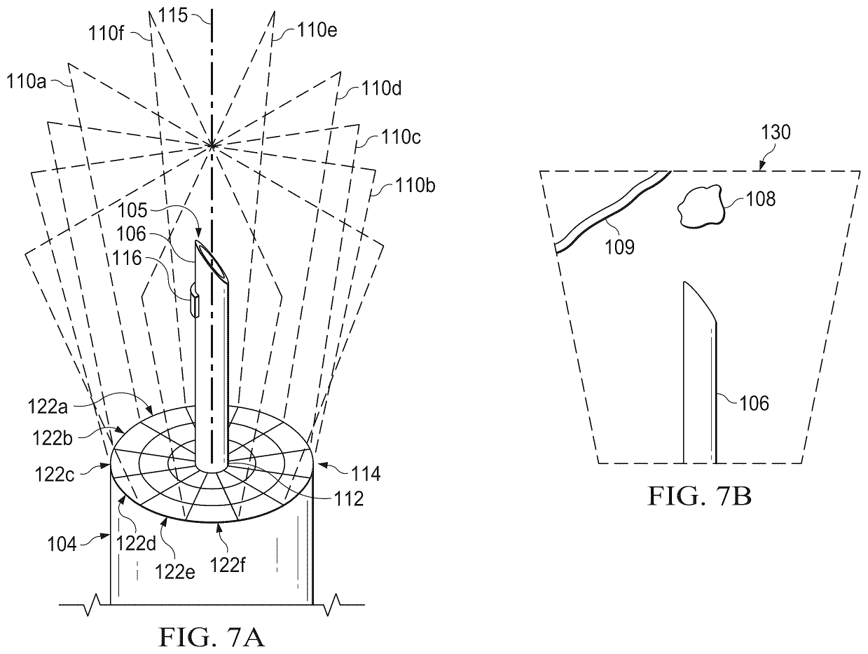

FIG. 7A illustrates a perspective view of a distal end of an imaging probe 104. In the illustrated example of FIG. 7A, the imaging probe 104 is substantially similar to the imaging probe of FIG. 6A but the concepts described in relation to FIGS. 7A-9B are similarly applicable to each example of an imaging probe described herein. The imaging array 114 in FIG. 7A is shown as having six potential imaging planes 110a-110f, one imaging plane for each column 122a-122f of imaging elements 118. However, it should be appreciated that various combinations of imaging elements 118 may be activated by a controller to generate images along additional potential imaging planes. In this regard, a greater number of potential imaging planes may be possible but for the sake of illustration, only six potential imaging planes 110a-110f are shown. The tool 106 is shown extended from the working channel 112 directly along the axis 115, as may occur with a rigid tool. Accordingly, all or a substantial portion of the tool 106, including the distal tip 105, would be visible in each of the six imaging planes 110a-110f.

FIG. 7B illustrates an example of an image 130 that may be captured along one of the imaging planes 110a-110f (e.g., 110a) during use of the imaging probe 104 with the tool 106 in the configuration shown in FIG. 7A. With the tool 106 positioned near the target tissue 108, in addition to the target tissue 108 and vasculature 109, the image 130 may capture a substantial longitudinal portion of the tool 106.

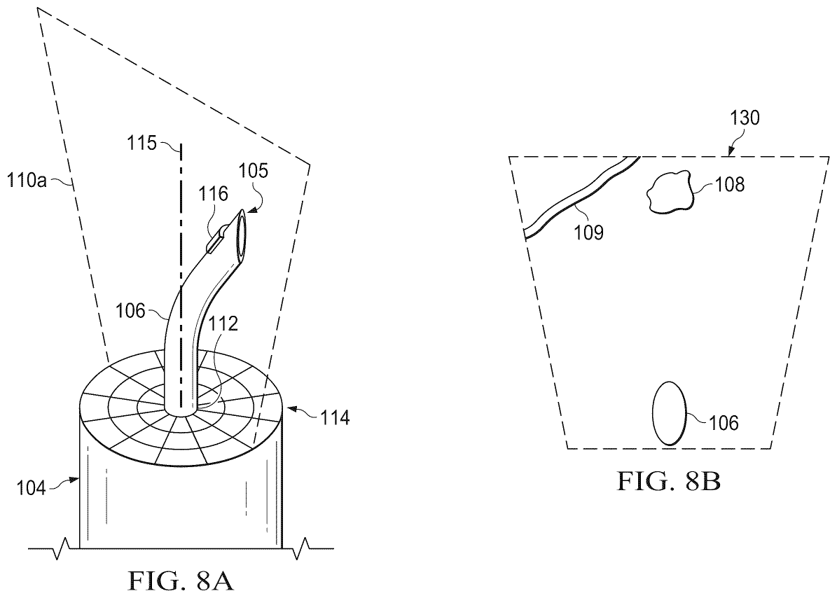

FIG. 8A illustrates a perspective view of the distal end of the imaging probe 104 of FIG. 7A with a tool 106 extending from the working channel 112 and bending away from the axis 115, as may occur with a flexible tool. Accordingly, a distal portion of the tool 106, including the distal tip 105, would not be visible in the imaging plane 110a or one or more other potential imaging planes. FIG. 8B illustrates an example of an image 130 that may be captured along imaging plane 110a during use of the imaging probe 104 with the tool 106 in the configuration shown in FIG. 8A. Although the target tissue 108 and vasculature 109 are visible, the image 130 may capture only a proximal longitudinal portion of the tool 106. Absent any additional information regarding the bent shape of the tool 106 or a position of the distal tip 105, a user may misinterpret the image 130 of FIG. 8B as showing the distal tip 105. Such a misinterpretation may result in the user continuing to advance the tool 106 distally from the working channel 112 to try to reach the target tissue 108. However, because the tool 106 is bent, the user may unintentionally advance the tool 106 in a direction that does not intersect the target tissue 108 and into tissue which may be critical to avoid, such as a portion of the vasculature 109 falling outside the imaging plane 110a. In order to provide the user with a more informative imaging plane, it is advantageous to determine a position of the distal tip 105, for example using the localization sensor 116 and select an imaging plane (e.g., one of imaging planes 110a-110f) that will illustrate a distal region of the tool 106 and/or other critical features of interest.

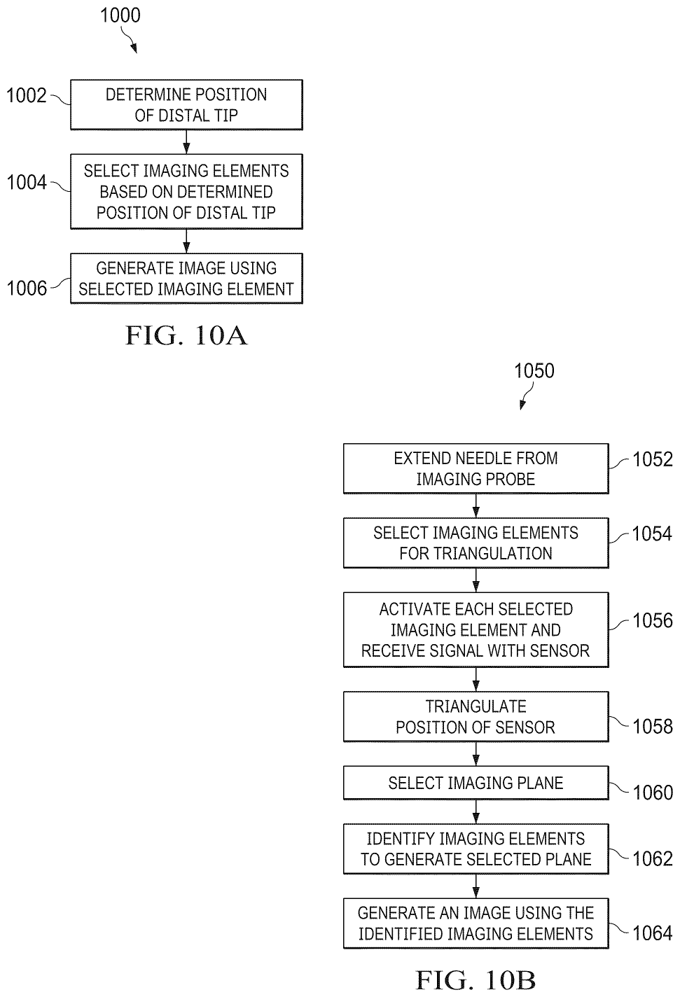

FIG. 9A illustrates the imaging probe 104 of FIG. 8A with the tool 106 in a similar bent configuration. The localization sensor 116 may be used to determine a position of the distal tip 105 relative to the imaging array 114 for the selection of an optimal imaging plane. A number of examples of a localization sensor are contemplated. For example, the localization sensor 116 may be a piezoelectric sensor (e.g., PZT sensor) in operative communication with a controller via an electrical wire (not shown) extending along a length of the tool 106 to the controller. For example, the wire may extend along an external surface of the tool 106, may be embedded within a wall of the tool, or more extend within an internal lumen of the tool. The piezoelectric sensor may be configured to detect an acoustic signal transmitted by one or more imaging elements. Alternatively, the localization sensor 116 may comprise an optical fiber extending along a length of the tool 106. A light source, such a laser, may transmit light through the optical fiber to Bragg gratings near the distal tip of the fiber. Light reflected back through the optical fiber from the distal tip may be analyzed by a sensor, such as a reflectometer, in communication with the controller. Signals transmitted by the imaging elements 118 may cause the wavelength of the light in the optical fiber to vary when they arrive at the distal tip of the optical fiber. As another alternative, the localization sensor may comprise a capacitance micromachined ultrasound transducer (“CMUT”) configured to receive an acoustic signal from the imaging elements 118 and/or transmit an acoustic signal to the imaging elements 118. In regard to the latter instance, it should be appreciated that the term “localization sensor” as used herein may refer to a transmitter configured to transmit a signal that is detectable by elements positioned on the imaging probe. Any other type of localization sensor may be used as is suitable for performing the techniques of the present disclosure. In some examples, the localization sensor 116 may comprise a WGM senor configured to measure pressure waves from a plurality of imaging elements 118 of the imaging array 114 to triangulate a location of the WGM sensor and, in turn, the distal tip 105 of the tool 106. The WGM sensor may be attached to an optical fiber having a known laser wavelength such that changes in wavelength may be detected and associated with the pressure waves. In an example, three or more imaging elements disposed around the outer edge of the imaging array may be activated for triangulating the location of the WGM sensor with respect to a known position of the activated imaging elements (e.g., their position within the imaging array and the position of the imaging array on the imaging probe 104. Use of the localization sensor 116 to determine the position of the distal tip 105 is described below with reference to FIG. 10B.

Once the position of the distal tip 105 has been determined relative to the imaging array, the controller may select an imaging plane that includes or is nearest to the distal tip 105. In the illustrated example of FIG. 9A, the imaging plane selected by the controller may be imaging plane 110c as shown in FIG. 7A. FIG. 9B illustrates an example of an image 130 that may be captured along imaging plane 110c during use of the imaging probe 104 with the tool 106 in the configuration shown in FIG. 9A. As shown, a substantial length of the tool 106 is visible in the image 130 because the imaging plane 110c crosses over the working channel 112 and intersects the tool 106 along most or all of its length as the tool is bent in one direction. It will be appreciated that a tool may bend in two orthogonal directions forming a more complex curvature which cannot be captured in a single imaging plane. Accordingly, the controller may select an imaging plane that captures a distal-most region of the tool and omits a more proximal region of the tool.

In the illustrated image 130 of FIG. 9B, the controller may determine that while most of the tool 106 is visible in-plane, the distal tip 105 may be out-of-plane. Accordingly, the controller may generate and display a virtual indicator 107 showing a projection of a position of the distal tip 105 with respect to the imaging plane. The virtual indicator 107 may include a numerical distance indication such as the number of millimeters between the distal tip 105 and the imaging plane being displayed. A direction indication may also be provided to convey whether the distal tip 105 is positioned proximally of the imaging plane (e.g., out of the page) or distally of the imaging plane (e.g., into the page). The direction indication may include, for example, a symbol, an arrow, or a color denoting the direction.

Although shown as having an arcuate shape, it will be appreciated that each imaging element 118 of FIGS. 7A, 8A, and 9A may have a square or rectangular shape, similar to the arrangement shown in FIG. 3C. Furthermore, the principles described and illustrated in relation to FIGS. 7A-9B are similarly applicable to each examples provided herein including those illustrated in FIGS. 2A-6B.

FIG. 10A provides a flowchart of an example of a method 1000 for generating an image. The method 1000 is illustrated as a set of operations or processes that may be performed in the same or in a different order than the order shown in FIG. 10A. One or more of the illustrated processes may be omitted in some examples of the method. Additionally, one or more processes that are not expressly illustrated in FIG. 10A may be included before, after, in between, or as part of the illustrated processes. In some examples, one or more of the processes of method 1000 may be implemented, at least in part, by a control system executing code stored on non-transitory, tangible, machine-readable media that when run by one or more processors (e.g., the processors of a control system) may cause the one or more processors to perform one or more of the processes.

After navigating an imaging probe (e.g., imaging probe 104) to a deployment location in the vicinity of a target tissue, at process 1002 a position of a distal tip of a tool (e.g., a tool 106 such as a needle) is determined. This may include any suitable method for determining the position of the distal tip including, but not limited to, triangulating the location of a localization sensor disposed on the tool, determining the position of an EM position sensor on the tool, capturing a plurality of images with different imaging planes and processing the images to identify the distal tip, etc. With regard to the latter, multiple or all of the possible combinations of imaging elements to capture different imaging planes may be used sequentially to capture an image in each plane. Image processing may then be performed to identify the optimal image that captures the distal tip, or other portion of interest, of the tool. Based on the known orientation of the imaging plane of the optimal image, the location of the distal tip can be determined from the optimal image. In this regard, a localization or EM sensor on the tool may not be necessary. In some examples, only a subset of all possible imaging planes may be used to determine the position of the distal tip. For example, an imaging probe having 20 possible imaging planes may utilize only a subset of 4 or 5 imaging planes. In the event the distal tip is not captured in one of the images of the subset of imaging planes, the position of the distal tip may be determined by interpolating between the imaging planes.

At a process 1004, the controller selects the imaging elements of an imaging array on the imaging probe that are to be used to generate an image that includes the distal tip of the tool. The selected imaging elements may be selected based on the determined position of the distal tip of the tool. The selected imaging elements may include all of the imaging elements of a particular imaging array or a subset thereof. For example, a single column of imaging elements extending across the imaging array may be selected (e.g., column 122a including imaging elements 118a-118f of FIG. 6B), two adjacent columns of imaging elements may be selected (e.g., for an imaging plane aligned with a gap between the adjacent columns), or a distributed subset of imaging elements across several columns and/or rows may be selected.

At a process 1006, the controller may proceed to generate the image using the selected imaging elements. If the generated image is determined to be unsatisfactory, for example if none or only a small portion of the tool is displayed, the controller may select a different subset of imaging elements and repeat the process. This selection of a different subset may be initiated automatically based on processing of the image data or in response to a user input.

As such, the controller controls at least a portion of the plurality of imaging elements to generate the image that includes an imaging plane that includes a position associated with a portion of the tool. The position may be the distal tip of the tool, or some other portion. The imaging elements may be controlled to only generate an image with the determined position of the portion of the tool. Alternatively, the imaging elements may be controlled to generate multiple images in different image planes and then an image processing may be performed to identify the image that includes the imaging plane that includes the position associated with the portion of the tool from the multiple images. In some embodiments, generating the image via the image processing may include generating a (e.g., 3D) model of the tool based on the multiple images in different imaging planes, identifying a position of a desired portion of the tool (e.g., distal tip) from the model, and selecting the image that includes the portion of the tool based on the identified position of the distal portion of the tool.

FIG. 10B provides another example of a method 1050 for generating an image, which may be similar to the method 1000. The method 1050 is illustrated as a set of operations or processes that may be performed in the same or in a different order than the order shown in FIG. 10B. One or more of the illustrated processes may be omitted in some examples of the method. Additionally, one or more processes that are not expressly illustrated in FIG. 10B may be included before, after, in between, or as part of the illustrated processes. In some examples, one or more of the processes of method 1050 may be implemented, at least in part, by a control system executing code stored on non-transitory, tangible, machine-readable media that when run by one or more processors (e.g., the processors of a control system) may cause the one or more processors to perform one or more of the processes.

After navigating the imaging probe (e.g., imaging probe 104) to a deployment location in the vicinity of a target tissue, at process 1052 a tool (e.g., a tool 106 such as a needle) is extended from a working channel of the imaging probe.

At process 1054, a controller in communication with the imaging array (e.g., imaging array 114) of the imaging probe selects a subset of imaging elements (e.g., imaging elements 118) of the imaging array for performing localization of a localization sensor (e.g., sensor 116) and, in turn, a distal tip (e.g., distal tip 105) of the tool by a triangulation process. In some examples, the subset of imaging elements may be a default subset of imaging elements used for all localization processes or the subset may be selected based on properties or characteristics of current procedure, for example, a type of tool being used, a flexibility of the tool, a distance by which the tool has been extended from the imaging probe, a type of tissue in which the target tissue is disposed, etc. If a tool is sufficiently rigid or the tissue is sufficiently soft, it may be acceptable to select a subset of only two imaging elements to determine the position of the localization sensor and distal tip. On the other hand, if the tool is substantially flexible, at least three imaging elements may be needed to accurately determine the position of the localization sensor and distal tip. With reference to FIG. 9A, the controller may select a subset of imaging elements including imaging elements 118a-118c. These particular imaging elements 118a-118c may be determined by the controller to be best suited for localization of the localization sensor 116 and distal tip 105 based on their positioning in the outer row of imaging elements (row 120a in FIG. 6B) and being spaced apart by approximately 120° apart. It will be appreciated for the following discussion that maximum spacing between the imaging elements selected for localization may be desirable to improve localization accuracy. In some examples, rather than using a subset of imaging elements of the imaging array that is used to generate images, additional elements (e.g., transducers) dedicated for localization may be disposed on the tool. For example, localization elements may be interspersed with the imaging elements of the imaging array or disposed about a perimeter of the imaging array.

At process 1056, the imaging elements of the selected subset are activated by the controller. The selected imaging elements may be activated sequentially in an order determined by the controller or in a default order. During process 1056, a first of the selected imaging elements may be “fired” to transmit an acoustic wave through the tissue between the imaging array and the localization sensor (e.g., along travel paths 128a-128c of FIG. 9A). Upon reaching the localization sensor, the localization sensor detects the acoustic wave and transmits a signal to controller indicating that the acoustic wave has been detected. The controller may record a time at which the imaging element is activated and a corresponding time at which the acoustic wave is detected by the localization sensor. The controller may then fire the next selected imaging element.

Alternatively, such as when the localization sensor comprises a CMUT, the controller may instruct the localization sensor to transmit a signal and may activate the selected imaging elements to detect the signal. A time at which the signal is transmitted may be recorded by the controller, as well as the time at which each respective imaging element or localization element received the signal.

Regardless of the direction in which the signal is sent between the imaging elements and the localization sensor, at a process 1058 the controller determines the position of the localization sensor with respect to the imaging array by triangulation using the time of travel of the respective signal between the selected imaging elements and the localization sensor. When the type of tissue through which the respective signals are transmitted is uniform, a longer time of travel indicates a longer distance between the localization sensor and an imaging element while a shorter time of travel indicates a shorter distance. When different types of tissue are disposed along different travel paths, the controller may adjust recorded travel times to compensate for different signal speeds through the different tissues. Using known properties regarding the speed at which a signal travels through the particular tissue(s), the controller may convert the travel times into distances. With the distances along each travel path being determined, the controller can determine the position of the localization sensor and, in turn, the position of the distal tip. In some examples, the controller may determine a position of the distal tip using the known fixed relationship between the distal tip and the localization sensor and, in some examples, the localization sensor may be disposed sufficiently close to the distal tip that the position of the distal tip may be assumed to be the position of the localization sensor.

With the position of the localization sensor and/or distal tip having been determined, at process 1060 the controller selects an imaging plane (e.g., imaging plane 110c of FIG. 9A). The selected imaging plane may be the potential imaging plane that is nearest to the localization sensor or distal tip of the tool. However, the controller may consider other features of interest in selecting an imaging plane. For example, the controller may account for the location of the target tissue and/or the location of vasculature or other tissue to be avoided. In this regard, the controller may select an imaging plane which includes the distal tip or a portion of the tool near the distal tip as well as one or more additional features of interest. For example, in some instances, the distal tip may be determined to be between two adjacent potential imaging planes. In selecting an imaging plane, the controller may determine one of the two adjacent potential imaging planes includes the target tissue while the other does not and, therefore, select the imaging plane that includes the target tissue.

At a process 1062, the controller identifies the imaging elements of the imaging array that are to be used to generate an image of the selected imaging plane. The identified imaging elements may include all of the imaging elements of a particular array or a subset thereof. For example, a single column of imaging elements extending across the imaging array may be identified (e.g., column 122a including imaging elements 118a-118f of FIG. 6B), two adjacent columns of imaging elements may be identified (e.g., for an imaging plane aligned with a gap between the adjacent columns), or a distributed subset of imaging elements may be identified. The controller determines the order in which the identified imaging elements should be activated and may proceed to generate the image at process 1064 using the identified imaging elements.

If the generated image is determined to be unsatisfactory, for example if only a small portion of the tool or target tissue is displayed, the controller may select a different subset of imaging elements. This selection of a different subset may be initiated automatically based on processing of the image data or in response to a user input.

Optionally, if the generated image indicates the tool has veered away from the target tissue or toward tissue to be avoided, the method 1050 may further include retracting the tool, repositioning the imaging probe, and repeating the processes 1052-1062. If the generated image indicates the tool is on an acceptable path to the target tissue, the processes 1054-1062 may be repeated.

In some examples, the techniques of this disclosure, such as those discussed in relation to FIGS. 10A-10B, may be used in a medical procedure performed with an imaging probe or catheter which may be hand-held or otherwise manually controlled. In other examples, these techniques may be used in a medical procedure performed with a robot-assisted medical system as shown in FIGS. 11-12. FIG. 11 illustrates a clinical system 10 that includes a robot-assisted medical system 1100. The robot-assisted medical system 1100 generally includes a manipulator assembly 1102 for operating a medical instrument system 1104 (including, for example, imaging probe 104 or catheter 124) in performing various procedures on a patient P positioned on a table T in a surgical environment 1101. The manipulator assembly 1102 may be robot-assisted, non-assisted, or a hybrid robot-assisted and non-assisted assembly with select degrees of freedom of motion that may be motorized and/or robot-assisted and select degrees of freedom of motion that may be non-motorized and/or non-assisted. A master assembly 1106, which may be inside or outside of the surgical environment 1101, generally includes one or more control devices for controlling manipulator assembly 1102. Manipulator assembly 1102 supports medical instrument system 1104 and may include a plurality of actuators or motors that drive inputs on medical instrument system 1104 in response to commands from a control system 1112. The actuators may include drive systems that when coupled to medical instrument system 1104 may advance medical instrument system 1104 into a naturally or surgically created anatomic orifice. Other drive systems may move the distal end of medical instrument system 1104 in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to actuate an articulable end effector of medical instrument system 1104 for grasping tissue in the jaws of a biopsy device and/or the like.

Robot-assisted medical system 1100 also includes a display system 1110 (which may display image 100 of FIG. 1) for displaying an image or representation of the surgical site and medical instrument system 1104 generated by a sensor system 1108 and/or an endoscopic imaging system 1109. Display system 1110 and master assembly 1106 may be oriented so operator O can control medical instrument system 1104 and master assembly 1106 with the perception of telepresence.

In some examples, medical instrument system 1104 may include components for use in surgery, biopsy, ablation, illumination, irrigation, or suction. Medical instrument system 1104, together with sensor system 1108 may be used to gather (i.e., measure) a set of data points corresponding to locations within anatomic passageways of a patient, such as patient P. In some examples, medical instrument system 1104 may include components of the endoscopic imaging system 1109, which may include an imaging scope assembly or imaging instrument (such as imaging probe 104) that records a concurrent or real-time image of a surgical site and provides the image to the operator or operator O through the display system 1110. The concurrent image may be, for example, a two or three-dimensional image captured by an imaging instrument positioned within the surgical site. In some examples, the endoscopic imaging system components may be integrally or removably coupled to medical instrument system 1104. However, in some examples, a separate endoscope, attached to a separate manipulator assembly may be used with medical instrument system 1104 to image the surgical site. The endoscopic imaging system 1109 may be implemented as hardware, firmware, software, or a combination thereof which interact with or are otherwise executed by one or more computer processors, which may include the processors of the control system 1112.

The sensor system 1108 may include a position/location sensor system (e.g., an electromagnetic (EM) sensor system) and/or a shape sensor system for determining the position, orientation, speed, velocity, pose, and/or shape of the medical instrument system 1104.

Robot-assisted medical system 1100 may also include control system 1112. Control system 1112 includes at least one memory 1116 and at least one computer processor 1114 for effecting control between medical instrument system 1104, master assembly 1106, sensor system 1108, endoscopic imaging system 1109, intra-operative imaging system 1118, and display system 1110. Control system 1112 (which may include a controller in operative communication with the imaging array 114 and/or localization sensor 116) also includes programmed instructions (e.g., a non-transitory machine-readable medium storing the instructions) to implement some or all of the methods described in accordance with aspects disclosed herein, including instructions for providing information to display system 1110.

Control system 1112 may further include a virtual visualization system to provide navigation assistance to operator O when controlling medical instrument system 1104 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like.

An intra-operative imaging system 1118 may be arranged in the surgical environment 1101 near the patient P to obtain images of the anatomy of the patient P during a medical procedure. The intra-operative imaging system 1118 may provide real-time or near real-time images of the patient P. In some examples, the intra-operative imaging system 1118 may comprise an ultrasound imaging system for generating two-dimensional and/or three-dimensional images. For example, the intra-operative imaging system 1118 may be at least partially incorporated into an imaging probe such as imaging probe 104. In this regard, the intra-operative imaging system 1118 may be partially or fully incorporated into the medical instrument system 1104.

FIG. 12 illustrates a surgical environment 1200 with an instrument reference frame (X_{I}, Y_{I}, Z_{I}) 250 in which the patient P is positioned on the table T. Patient P may be stationary within the surgical environment in the sense that gross patient movement is limited by sedation, restraint, and/or other means. Cyclic anatomic motion including respiration and cardiac motion of patient P may continue unless the patient is asked to hold his or her breath to temporarily suspend respiratory motion. Within surgical environment 1200, an elongate instrument 1214 (e.g., a portion of the medical instrument system 1104 such as an imaging probe 104 or a catheter 124) is coupled to an instrument carriage 1206. In this example, elongate instrument 1214 includes an elongate instrument 1202 coupled to an instrument body 1212. Instrument carriage 1206 is mounted to an insertion stage 1208 fixed within surgical environment 1200. Alternatively, insertion stage 1208 may be movable but have a known location (e.g., via a tracking sensor or other tracking device) within surgical environment 1200. In these alternatives, the instrument reference frame 250 is fixed or otherwise known relative to the surgical reference frame. Instrument carriage 1206 may be a component of a robot-assisted manipulator assembly (e.g., robot-assisted manipulator assembly 1102) that couples to elongate instrument 1214 to control insertion motion (i.e., motion along an axis A) and motion of a distal end 1218 of the elongate instrument 1202 in multiple directions including yaw, pitch, and roll. Instrument carriage 1206 or insertion stage 1208 may include actuators, such as servomotors, (not shown) that control motion of instrument carriage 1206 along insertion stage 1208.

As shown in FIG. 12, instrument body 1212 is coupled and fixed relative to instrument carriage 1206. In some examples, the optical fiber shape sensor 1204 is fixed at a proximal point 1216 on instrument body 1212. In some examples, proximal point 1216 of optical fiber shape sensor 1204 may be movable along with instrument body 1212 but the location of proximal point 1216 may be known (e.g., via a tracking sensor or other tracking device). Shape sensor 1204 measures a shape from proximal point 1216 to another point such as distal end 1218 of elongate instrument 1202 in the instrument reference frame (X_{I}, Y_{I}, Z_{I}) 250.

Elongate instrument 1202 includes a channel (not shown) sized and shaped to receive a medical instrument 1210. In some examples, medical instrument 1210 may be used for procedures such as surgery, biopsy, ablation, illumination, irrigation, or suction. Medical instrument 1210 can be deployed through elongate instrument 1202 and used at a target location within the anatomy. In an example in which elongate instrument 1202 comprises a catheter (e.g., catheter 124), medical instrument 1210 may include, for example, an imaging probe. In an example in which elongate instrument 1202 comprises an imaging probe (e.g., imaging probe 104), medical instrument 1210 may include a biopsy instrument, laser ablation fiber, and/or other surgical, diagnostic, or therapeutic tool. Medical instrument 1210 may be advanced from the distal end 1218 of the elongate instrument 1202 to perform the procedure and then retracted back into the channel when the procedure is complete. Medical instrument 1210 may be removed from proximal end of elongate instrument 1202 or from another instrument port (not shown) along elongate instrument 1202.

Elongate instrument 1202 may also house cables, linkages, or other steering controls (not shown) to controllably bend distal end 1218. In some examples, at least four cables are used to provide independent “up-down” steering to control a pitch of distal end 1218 and “left-right” steering to control a yaw of distal end 1218.

A position measuring device 1220 provides information about the position of instrument body 1212 as it moves on insertion stage 1208 along an insertion axis A. Position measuring device 1220 may include resolvers, encoders, potentiometers, and/or other sensors that determine the rotation and/or orientation of the actuators controlling the motion of instrument carriage 1206 and consequently the motion of instrument body 1212. In some examples, insertion stage 1208 is linear, while in other examples, the insertion stage 1208 may be curved or have a combination of curved and linear sections.

Advantages of the present disclosure will be appreciated and include direct visualization providing confirmation of a tool such as a needle being inserted into a target tissue and avoiding a hazard such as vasculature even as the tool bends out of an imaging plane. In this regard, if the tip of a needle veers out of an imaging plane during insertion, a different imaging plane can be selected and a new image can be generated. Further, the systems and methods described herein provide assurance that a portion of a tool that is visible in an image is the distal tip or is at least near the distal tip. Moreover, the described techniques provide reduced image processing latency. Rather than firing all of the imaging elements through all imaging planes in an attempt to locate the distal tip of the tool, the position is determined and only the imaging elements needed to produce the optimal imaging plane may be fired. It should also be appreciated that annular imaging arrays for a forward-facing approach may provide direct alignment of the imaging probe to the target tissue and there may be less frictional resistance to deployment of the tool as compared to a side-facing approach in which the tool may be substantially bent to exit through a side port.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one example may be combined with the features, components, and/or steps described with respect to other examples of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

The methods described herein are illustrated as a set of operations or processes. Not all the illustrated processes may be performed in all examples of the methods. Additionally, one or more processes that are not expressly illustrated or described may be included before, after, in between, or as part of the example processes. In some examples, one or more of the processes may be performed by the control system (e.g., control system 1112) or may be implemented, at least in part, in the form of executable code stored on non-transitory, tangible, machine-readable media that when run by one or more processors (e.g., the processors 1114 of control system 1112) may cause the one or more processors to perform one or more of the processes.

Any described “imaging device” herein may include an ultrasound array, optical imaging device, or any other suitable imaging hardware. Any described “imaging probe” may include an ultrasound probe, an optical imaging probe, or a probe incorporating any other suitable imaging modality. Additionally, any “ultrasound array,” “imaging array,” or “imaging device” as described herein may comprise a single imaging element (e.g., transducer) or a plurality of such devices.

One or more elements in examples of this disclosure may be implemented in software to execute on a processor of a computer system such as control processing system. When implemented in software, the elements of the examples of the invention are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In one example, the control system supports wireless communication protocols such as Bluetooth, IrDA, HomeRF, IEEE 802.11, DECT, and Wireless Telemetry.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the invention are not described with reference to any particular programming language. It will be appreciated that a variety of programming languages may be used to implement the teachings of the invention as described herein.

In some instances well known methods, procedures, components, and circuits have not been described in detail so as not to unnecessarily obscure aspects of the examples. This disclosure describes various instruments, portions of instruments, and anatomic structures in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

While certain exemplary examples of the invention have been described and shown in the accompanying drawings, it is to be understood that such examples are merely illustrative of and not restrictive on the broad invention, and that the examples of the invention not be limited to the specific constructions and arrangements shown and described, since various other modifications may occur to those ordinarily skilled in the art.

1. A system comprising:
a flexible elongate device comprising:
a working channel extending to an opening in a distal portion of the flexible elongate device; and
a plurality of imaging elements;
a tool extendable through the working channel and the opening; and
a controller comprising one or more processors configured to:
determine a position associated with a portion of the tool; and
control at least a portion of the plurality of imaging elements to generate an image that includes an imaging plane that includes the position associated with the portion of the tool.

2. (canceled)

3. The system of claim 1, wherein determining the position associated with the portion of the tool comprises:
capturing a plurality of images using different subsets of the plurality of imaging elements, each image of the plurality of images having a different imaging plane; and
processing the images to identify the portion of the tool within one or more images of the plurality of images.

4. The system of claim 1, wherein the plurality of imaging elements includes:
an annular array of the imaging elements disposed around the opening, the annular array including at least two rows of imaging elements; or
a grid array in which the plurality of imaging elements are arranged in at least two rows and at least two columns.

5. The system of claim 1, wherein the plurality of imaging elements includes:
an array of ultrasound transmitters configured to generate ultrasound waves and an array of ultrasound sensors configured to detect reflected ultrasound waves; or
an array of transducers configured to generate and detect ultrasound waves.

6-8. (Canceled)

9. The system of claim 1, wherein:
the plurality of imaging elements is configured to capture images including imaging planes defined within an imaging field located distally of the distal portion of the flexible elongate device; and
the working channel extends through a distal end surface of the flexible elongate device to direct the tool into the imaging field.

10. The system of claim 1, wherein:
the plurality of imaging elements is configured to capture images including imaging planes defined within in an imaging field located radially outward from an outer side surface of the flexible elongate device; and
the working channel extends through a side wall of the flexible elongate device to direct the tool into the imaging field.

11. (canceled)

12. The system of claim 1, wherein:
the controller is further configured to determine a second position associated with one of a second position of the tool or a region of interest in a target tissue; and
the imaging plane includes both the position and the second position.

13. The system of claim 1, wherein the flexible elongate device further comprises a balloon configured for inflation with a fluid for acoustic transmission between the plurality of imaging elements and tissue.

14. (canceled)

15. The system of claim 1, wherein the portion of the tool includes a distal tip of the tool and at least a section of the tool is flexible.

16. The system of claim 1, wherein the tool includes a localization sensor located at the portion of the tool.

17. The system of claim 16, wherein the localization sensor comprises a fiber optic sensor configured to detect a change in a wavelength of light in a fiber in response to pressure waves generated by the plurality of imaging elements.

18. The system of claim 16, wherein the localization sensor comprises:
a WGM resonator;
a piezoelectric sensor; or
a capacitance micromachined ultrasound transducer.

19-20. (canceled)

21. The system of claim 16, wherein the controller is configured to determine the position of the portion of the tool by:
sequentially activating a subset of the plurality of imaging elements to transmit pressure waves;
detecting a pressure wave from each imaging element of the subset;
determining a time of travel of the pressure wave from each imaging element of the subset to the localization sensor; and
triangulating the position of the portion of the tool based on the time of travel of each pressure wave from the respective imaging element to the localization sensor.

22. (canceled)

23. The system of claim 16, wherein the controller is configured to determine the position of the portion of the tool by:
activating the localization sensor to transmit a pressure wave;
activating a subset of the plurality of imaging elements to detect the pressure wave;
determining a time of travel of the pressure wave from the localization sensor to each imaging element of the subset; and
triangulating the position of the portion of the tool based on the time of travel of the pressure wave from the localization sensor to each imaging element of the subset.

24. The system of claim 1, wherein the controller is configured to control the at least a portion of the plurality of imaging elements to generate the image by determining a firing sequence of the at least a portion of the plurality of imaging elements based on the imaging plane.

25. (canceled)

26. The system of claim 1, wherein the controller is further configured to:
associate sets of imaging elements with different image planes;
determine the imaging plane as being nearest to the position associated with the portion of the tool; and
generate the image using a set of imaging elements associated with the determined imaging plane.

27. (canceled)

28. The system of claim 1, wherein at least a portion of the tool includes a surface enhancement to alter reflection of an ultrasound wave from the plurality of imaging elements.

29. A method for capturing an image performed by a controller comprising one or more processors, the method comprising:
determining a position associated with a portion of a tool at least partially located in a working channel of a flexible elongate device, the working channel extending to an opening in a distal portion of the flexible elongate device; and
controlling at least a portion of a plurality of imaging elements of the flexible elongate device to generate an image that includes an imaging plane that includes the position associated with the portion of the tool.

30. (canceled)

31. The method of claim 29, wherein determining the position associated with the portion of the tool comprises:
capturing a plurality of images using different subsets of the plurality of imaging elements, each image of the plurality of images having a different imaging plane; and
processing the images to identify the portion of the tool within one or more images of the plurality of images.

32. The method of claim 29, further comprising:
displaying a virtual indicator providing a visual representation of a position of the portion of the tool with respect to the imaging plane.

33-35. (canceled)
